(19) United States
(12) Patent Application Publication
(10) Pub. No.: US 2026/0272351 A1
(43) Pub. Date: Sep. 17, 2026

(54) A METHOD FOR PROCESSING A TEXT FOR CLASSIFYING ONE OR MORE INDIVIDUALS IN ONE OR MORE CATEGORIES

(71) Applicant: MYNDOOR S.R.L., Rosate (MI) (IT)

(72) Inventors: Francesco FINAZZI, Rosate (MI) (IT); Matteo MENDULA, Bologna (BO) (IT)

(21) Appl. No.: 19/469,156

(22) PCT Filed: Mar. 27, 2024
(86) PCT No.: PCT/IB2024/052963

§ 371 Date: Sep. 25, 2025

(30) Foreign Application Priority Data
Mar. 28, 2023 (IT) 102023000005958

(51) Int. Cl. A61B 5/16; G06F 40/279; G06F 40/30

(52) U.S. Cl. CPC A61B 5/165; G06F 40/279; G06F 40/30

(57) ABSTRACT

A method for processing a text, in order to classify one or more individuals in one or more categories characterized by specific mental conditions, comprises the following steps, performed by a processor:
receiving (F1) a text string (ST);
removing (F2) the insignificant characters from the text string (ST) to define a clean text string (STP);
subdividing (F3) the text string into the respective words (P);
associating (F4) each word with a respective category, associable with a mental condition of the individual.
   The step of associating each word (P) with the respective category is performed by a machine-learned model.

### TECHNICAL FIELD

This invention relates to a method for processing a text in a method for classifying one or more individuals in one or more categories characterized by specific mental conditions.

### BACKGROUND ART

Known in the prior art in the field of classification used as a method for classifying one or more individuals based on strings of text correlated with them, are systems which, based on the words included in the text, calculate a score for each individual with respect to the class, so as to classify the individual adequately in the class if they exceed a certain score. To do this, text processing logics are used which, however, are poorly performing and subject to significant classification errors.

For example, such text processing methods tend to assign scores to the words without taking into account some important aspects which could completely change the significance of the headwords and lead to results which are totally wrong.

### Disclosure of the Invention

The aim of this disclosure is to provide a method for processing a text and a related computer program which overcome the above-mentioned drawbacks of the prior art.

This aim is fully achieved by the method and computer program of this disclosure as characterized in the appended claims.

According to an aspect of it, this disclosure relates to a method for processing a text in a method for classifying one or more individuals in one or more categories characterized by specific mental conditions. The method is carried out by a processor.

The method comprises a step of receiving a text string. The method comprises a step of removing insignificant characters from the text string. The method comprises a step of dividing the text string into respective headwords (or words).

The method comprises a step of associating each word with a respective category, associable with a mental condition of the individual.

Preferably, the step of associating each word with the respective category is performed by a machine-learned model.

In particular, the model is trained by means of a database which associates each headword with a corresponding significance with respect to a class, representing a specific mental condition. After being trained, therefore, the machine-learned model is capable of associating a score with each headword.

Using a machine-learned model allows a more precise classification of the individuals which the normal prior art methods are unable to provide. In an embodiment, the machine-learned model calculates and associates with each word a respective score, which identifies the significance of the word with respect to the mental condition associated with the individual.

In an embodiment, the method comprises a step of subdividing the text string into semantic groups. Each of the semantic groups represents a portion of the text string associated with a respective topic. The machine-learned model associates a respective score with each word based on the semantic group the word belongs to.

In other words, by analysing the headwords, the model is able to understand the topic at hand. Based on the topic, the headwords have a varying degree of relevance to a specific mental condition.

Specifically, the machine-learned model associates a relevance value with each semantic group. This is done on the basis of a topic database including a respective relevance value for each semantic group.

The machine-learned model associates a respective score with each word based on the relevance value of the semantic group the word belongs to.

In other words, once the topic is identified and the corresponding relevance value retrieved, the relevance value contributes to calculating (with different computational methods) the score of the headwords belonging to the semantic group.

The machine-learned model performs a step of identifying, among the words of the text string, a first word which semantically represents an adversative conjunction. Next, it searches, among the words following the first word, for a second word whose score is higher than a threshold value. In other words, the model assesses the proximity between an adversative conjunction and a word (a headword) having a certain significance for the purposes of classification (thus having a minimum score).

The method comprises a step of identifying an effective inversion of text polarity if the step of searching for the second word returns a positive result.

This method is very important because it allows relevantly assessing the presence of the adversative conjunctions, accordingly varying the result of the final classification. In effect, many methods do not include this type of analysis and simply ignore the adversative conjunctions or vary the classification even when the adversative does not have a true value of relevant polarity inversion.

The machine-learned model recognizes the presence of an exclamation mark associated with a group of words of the text string. The machine-learned model calculates the score based on the presence of the exclamation mark, so that the presence of the exclamation mark increases the score of the words in the group.

Indeed, when classifying into specific classes characterized by mental conditions, the exclamation mark has a very high significance which is not at all relevant in other applications.

In an embodiment, it is preferable that, of the headwords preceding the question mark, the machine-learned model identifies the one with the highest score (highest significance) and consequently increases its score. If there is more than one headword with a high score, the machine-learned model modifies the score of all these headwords accordingly.

The machine-learned model associates a higher score with the words that represent adjectives compared to words that represent nouns. In effect, based on the numerous experiments and tests conducted, it was observed that the precision of the method increases if adjectives are taken into greater consideration than nouns.

In an embodiment, the machine-learned model varies the score of a word based on whether that word is preceded or followed by words that indicate intensity modifiers, for example, adverbs.

This aspect, too, is absolutely relevant for such a classification because, while adverbs are not relevant in some applications, they are, in this case, important modifiers of the indications regarding the mental state of the individuals and thus influence the classification.

It is a good idea to specify that the present method is not a rule driven method but a machine-learned model where knowledge injection strategies are used to calculate more accurately the weights to be used in the method. Therefore, compared to machine-learned models, the knowledge injection strategies allow increasing the reliability of the method.

The insignificant characters removed in the step of removing represent one or more of the following characters:
Internet addresses;
punctuation marks;
proper nouns of people and/or towns or cities;
prepositions;
non-adversative conjunctions.

Preferably, the machine-learned model is a supervised artificial intelligence algorithm comprising a self-learning module.

This clearly allows progressively improving the identification of the correct score for each headword in its semantic context.

In an embodiment, the machine-learned model, based on the scores associated with the words (headwords) of each text string, classifies the individual as falling into one or more mental conditions. Alternatively, since two or more mental conditions can coexist, the machine-learned model determines, for each of the coexisting mental conditions, a probability that the individual can fall into it.

Furthermore, again based on the scores assigned to the words, the machine-learned model may, for each mental condition which the individual falls into, also determine a corresponding degree of severity.

According to an aspect of it, this disclosure provides a computer program, including instructions for executing one or more of the steps of the method according to any of the aspects described above.

### BRIEF DESCRIPTION OF THE DRAWINGS

This and other features will become more apparent from the following description of a preferred embodiment, illustrated by way of non-limiting example in the accompanying drawing, in which:

FIG. 1 shows a schematic flow diagram representing the steps of a method for classifying individuals into one or more classes.

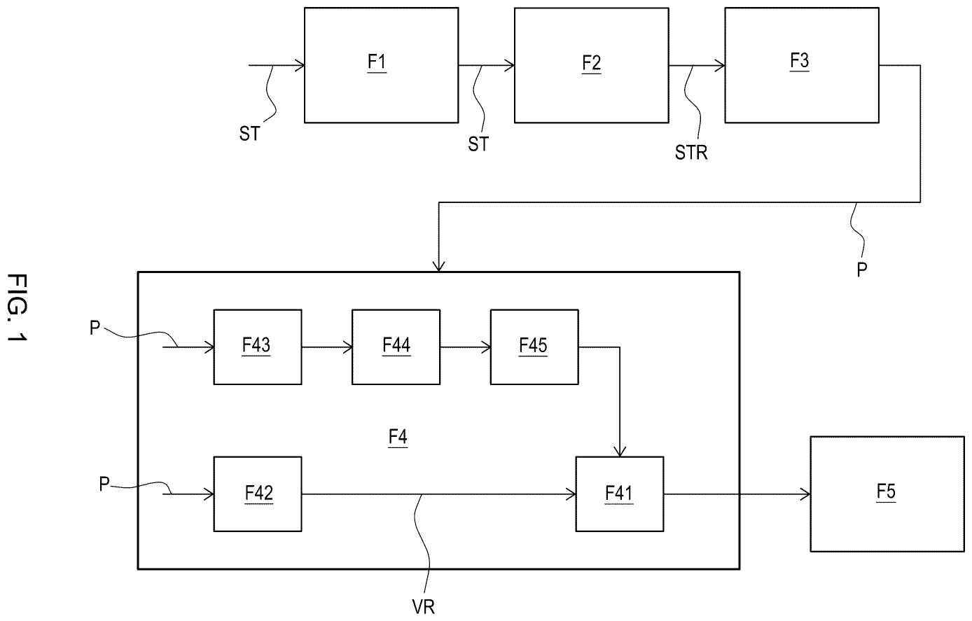

### DETAILED DESCRIPTION OF PREFERRED EMBODIMENTS OF THE INVENTION

As shown in the accompanying drawing, the method comprises a step of receiving F1 a text string ST, consisting, for example, of messages, texts, letters or any text produced by one or more individuals. The text string ST is cleared of (by a step of removing F2) the characters which are not significant for analysing the mental conditions of the individual in that, for example, they do not represent a frame of mind. For example, Internet addresses, punctuation marks, proper nouns of people and/or towns or cities, and/or prepositions are removed because they are words that do not indicate the individual's frame of mind. This allows creating a clean text string STP.

The method comprises a step of dividing F3 the clean text string STR into respective headwords P (or words).

The method comprises a step of associating F4 each word with a respective category, associable with a mental condition of the individual.

The step of associating F4 each word with the respective category is performed by a machine-learned model.

In an embodiment, the machine-learned model calculates and associates F41 with each headword P a respective score, which identifies the significance of the headword P with respect to the mental condition associated with the individual.

In an embodiment, the method comprises a step of subdividing F42 the text string into semantic groups. Each of the semantic groups represents a portion of the text string ST associated with a respective topic. The machine-learned model associates a respective score with each headword P based on the semantic group the headword P belongs to.

Specifically, the machine-learned model associates a relevance value with each semantic group. This is done on the basis of a topic database including a respective relevance value VR for each semantic group.

The machine-learned model associates a respective score with each headword P based on the relevance value VR of the semantic group the headword P belongs to.

The machine-learned model performs a step of identifying F43, among the words of the clean text string STR, a first headword P which semantically represents an adversative conjunction. Next, it searches F44 among the headwords following the first headword, for a second headword whose score is higher than a threshold value. In other words, the model assesses the proximity between an adversative conjunction and a word (a headword) having a certain significance for the purposes of classification (thus having a minimum score).

The method comprises a step of identifying F45 an effective inversion of text polarity if the step of searching for the second headword returns a positive result.

In an embodiment, the machine-learned model, based on the scores associated with the words (headwords) P of each text string ST, classifies F5 the individual as falling into one or more mental conditions.

In an embodiment, the method comprises a step of reinforcement learning in which a user can confirm or vary the category identified on the basis of the text input, so as to progressively improve the machine-learned model.

We note that, preferably, the individual's mental condition is a stress level of that individual. The stress level may be categorized based on the degree of stress: for example, high, medium, low.

It should be noted that in the step of training the machine-learned model, the method comprises feeding results of one or more questionnaires in association with corresponding text inputs of the individuals. In the field of stress assessment, such questionnaires are highly diversified and using a single questionnaire for training the machine-learned model carries some risk.

In this regard, the method comprises a step of ensembling, in which the results of the questionnaires are combined in such a way as to balance the errors and imprecisions of the single scores. The different number of questions, the different scales usable by the patient for their answers and the assessment of the times for filling out the questionnaire have led to the creation of a first machine-learned model which weighs the scores of the single questionnaires differently with the aim of enhancing the capabilities of the model in terms of accuracy and precision. Thus, in an embodiment, training the machine learning model comprises the following steps:
handing out a first questionnaire, including a respective different number of questions, respective different scales usable by the patient for their answers and respective preset times for filling out the questionnaire;
calculating a first score of the first questionnaire;
handing out a second questionnaire, including a respective different number of questions, respective different scales usable by the patient for their answers and respective preset times for filling out the questionnaire;
calculating a second score for a second questionnaire;
calculating a final score, based on the first and the second score as a function of the respective questions, scales and filling out times;
loading the text input in association with the final score calculated.

The score corresponds to a certain stress level.

It is also important to observe that this invention also involved human intervention for associating each text input with a score determined by a jury of one or more experts in the field. This allowed evaluating the stress level for each sentence.

Starting from these evaluations of a set of manually labelled sentences, the method comprises a step of distance labelling, that is to say, an automatic labelling process based on the results of a questionnaire for a user, which allows labelling a huge amount of data automatically so as to be able to train the machine-learned model with a huge amount of data but without manual labelling by the experts.

For example, if the stress level of a user has been assessed as being high from the questionnaires (or the ensembling of the questionnaires), then the method comprises labelling the user's statements with the same stress level. This involves a large increase in data, hence also in the reliability of the method but a reduction in manual efforts.

According to another formulation of this invention, this method comprises one or more of the following steps:
1. collecting text inputs through clinical trials;
2. filling out one or more stress questionnaires;
3. labelling the text inputs by the jury of experts;
4. first step of training the algorithm, in order to study the correlation between the questionnaire results and the related stress level;
5. based on the first step of training, determining the stress level based solely on the questionnaires and expanding the training data with data (defined as synthetic) obtained through the distance labelling process described above;
6. second step of training the algorithm;
7. generating new text inputs, obtained through the second step of training the algorithm, to further augment the training data;
8. cleaning the input data by removing internet addresses and punctuation marks;
9. correcting typos by calculating the similarity with similar words and replacing the incorrect words with the more similar correct ones;
10. third step of training the algorithm by supervised training of a customized LLM (Large Language Model) for determining the stress level, wherein the third step of training comprises the following steps:
   a. dividing the text input into bigrams and/or trigrams;
   b. calculating the polarity of a sentence through adversative conjunctions;
   C. increasing the weight given to the words near exclamation marks;
   d. increasing the weight given to adjectives compared to nouns;
   e. increasing the weight given to words near intensity modifiers such as extremely, very, particularly;
   f. increasing the weight given to words which have a high score for negative feelings such as: anxiety, sadness, disgust, surprise and fear (inferred through sentiment analysis);
11. determining the stressed or non-stressed semantic context regarding the input content;
12. evaluating the performance of the machine-learned model;
13. iteratively repeating steps 1-12.

1. A method for processing a text in a method for classifying one or more individuals in one or more categories characterized by specific mental conditions, the method comprising the following steps, performed by a processor:
receiving a text string;
removing the insignificant characters from the text string to define a clean text string;
subdividing the text string into the respective words;
associating each word with a respective category, associable with a mental condition of the individual,
wherein the step of associating each word with the respective category is performed by a machine-learned model.

2. The method according to claim 1, wherein, for each word, the machine-learned model performs a step of calculating a respective score, which identifies the significance of the word with respect to the mental condition associated with the individual.

3. The method according to claim 2, comprising a step of subdividing the text string into semantic groups, each representing a portion of the text string associated with a respective topic,
wherein the machine-learned model associates a respective score with each word based on the semantic group the word belongs to.

4. The method according to claim 3, wherein the machine-learned model associates a relevance value with each semantic group, based on a topic database including a respective relevance value for each semantic group, wherein the machine-learned model associates a respective score with each word based on the relevance value of the semantic group the word belongs to.

5. The method according to claim 2, wherein the machine-learned model performs the following steps:
identifying, among the words of the text string, a first word which semantically represents an adversative conjunction;
searching, among the words following the first word, for a second word whose score is higher than a threshold value;
identifying an effective inversion of text polarity if the step of searching for the second word returns a positive result.

6. The method according to claim 2, wherein the machine-learned model recognizes the presence of an exclamation mark associated with a group of words of the text string and calculates the score based on the presence of the exclamation mark, so that the presence of the exclamation mark increases the score of the words in the group.

7. The method according to claim 2, wherein the machine-learned model associates a higher score with the words that represent adjectives compared to words that represent nouns.

8. The method according to claim 2, wherein the machine-learned model varies the score of a word based on whether that word is preceded or followed by words that indicate intensity modifiers.

9. The method according to claim 1, wherein, in the step of removing, the insignificant characters represent one or more of the following characters:
Internet addresses;
punctuation marks;
proper nouns of people and/or towns or cities;
prepositions;
non-adversative conjunctions.

10. The method according to claim 1, wherein the machine-learned model is a supervised artificial intelligence algorithm comprising a self-learning module.

11. The method according to claim 2, wherein the machine-learned model performs the following steps, based on the scores associated with the words:
classifying the individual within one or more mental conditions, and/or
determining a probability of the individual being classified as falling into each of the mental conditions provided; and/or
determining a degree of severity of one or more mental conditions.

12. A computer program, including instructions for executing one or more of the steps of the method according to claim 1.
